SEQUENCE LISTING (6 sequences)

SEQ ID NO: 1 - protein, 430 aa, Artificial sequence
  JNK3 Protein sequence
MSLHFLYYCSEPTLDVKIAFCQGFDKQVDVSYIAKHYNMSKSKVDNQFYSVEVGDSTFTVLKRYQNLKPIGSGAQGIVCAAYDAVLDRNVAIKKLSRPFQNQTHAKRAYRELVLMKCVNHKNIISLLNVFTPQKTLEEFQDVYLVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDIWSVGCIMGEMVRHKILFPGRDYIDQWNKVIEQLGTPCPEFMKKLQPTVRNYVENRPKYAGLTFPKLFPDSLFPADSEHNKLKASQARDLLSKMLVIDPAKRISVDDALQHPYINVWYDPAEVEAPPPQIYDKQLDEREHTIEEWKELIYKEVMNSEEKTKNGVVKGQPSPSAQVQQDYKDDDDK

SEQ ID NO: 2 - protein, 246 aa, Artificial Sequence
  GFP protein sequence
MSKGEELFTGVVPILVELDGDVNGQKFSVSGEGEGDATYGKLTLKFICTTGKLPVPWPTLVTTFSYGVQCFSRYPDHMKQHDFFKSAMPEGYVQERTIFYKDDGNYKTRAEVKFEGDTLVNRIELKGIDFKEDGNILGHKMEYNYNSHNVYIMADKPKNGIKVNFKIRHNIKDGSVQLADHYQQNTPIGDGPVLLPDNHYLSTQSALSKDPNEKRDHMILLEFVTAAGITHGMDELYKDYKDDDDK

SEQ ID NO: 3 - DNA, 1290 bp, Artificial Sequence
  JNK3 protein coding sequence before optimization
atgagcctccatttcttatactactgcagtgaaccaacattggatgtgaaaattgccttttgtcagggattcgataaacaagtggatgtgtcatatattgccaaacattacaacatgagcaaaagcaaagttgacaaccagttctacagtgtggaagtgggagactcaaccttcacagttctcaagcgctaccagaatctaaagcctattggctctggggctcagggcatagtttgtgccgcgtatgatgctgtccttgacagaaatgtggccattaagaagctcagcagaccctttcagaaccaaacacatgccaagagagcgtaccgggagctggtcctcatgaagtgtgtgaaccataaaaacattattagtttattaaatgtcttcacaccccagaaaacgctggaggagttccaagatgtttacttagtaatggaactgatggatgccaacttatgtcaagtgattcagatggaattagaccatgagcgaatgtcttacctgctgtaccaaatgttgtgtggcattaagcacctccattctgctggaattattcacagggatttaaaaccaagtaacattgtagtcaagtctgattgcacattgaaaatcctggactttggactggccaggacagcaggcacaagcttcatgatgactccatatgtggtgacacgttattacagagcccctgaggtcatcctggggatgggctacaaggagaacgtggatatatggtctgtgggatgcattatgggagaaatggttcgccacaaaatcctctttccaggaagggactatattgaccagtggaataaggtaattgaacaactaggaacaccatgtccagaattcatgaagaaattgcaacccacagtaagaaactatgtggagaatcggcccaagtatgcgggactcaccttccccaaactcttcccagattccctcttcccagcggactccgagcacaataaactcaaagccagccaagccagggacttgttgtcaaagatgctagtgattgacccagcaaaaagaatatcagtggacgacgccttacagcatccctacatcaacgtctggtatgacccagccgaagtggaggcgcctccacctcagatatatgacaagcagttggatgaaagagaacacacaattgaagaatggaaagaacttatctacaaggaagtaatgaattcagaagaaaagactaaaaatggtgtagtaaaaggacagccttctccttcagcacaggtgcagcaggactacaaggatgatgatgacaaa

SEQ ID NO: 4 - DNA, 738 bp, Artificial Sequence
  GFP protein coding sequence before optimization
atgagtaaaggagaagaacttttcactggagttgtcccaattcttgttgaattagatggcgatgttaatgggcaaaaattctctgtcagtggagagggtgaaggtgatgcaacatacggaaaacttacccttaaatttatttgcactactgggaagctacctgttccatggccaacacttgtcactactttctcttatggtgttcaatgcttttcaagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaactataactcacataatgtatacatcatggcagacaaaccaaagaatggaatcaaagttaacttcaaaattagacacaacattaaagatggaagcgttcaattagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctgggattacacatggcatggatgaactatacaaagactacaaagatgatgatgacaag

SEQ ID NO: 5 - DNA, 1290 bp, Artificial Sequence
  JNK3 protein coding sequence after optimization
atgtctctgcacttcctgtactactgttctgagcccaccctggacgtgaagattgccttctgccagggctttgacaagcaggtggatgtgagctacatcgccaagcactacaacatgtccaagagcaaggtggacaaccagttctacagcgtggaggtgggagacagcaccttcacagtgctgaagagataccagaacctgaagccaattggctctggagcccagggcattgtgtgtgctgcctatgatgctgtgctggacagaaatgtggccatcaagaagctgagcagacccttccagaaccagacacatgccaagagagcctacagagagctggtgctgatgaagtgtgtgaaccacaagaacatcatcagcctgctgaatgtgttcacccctcagaagacactggaggagttccaggatgtgtacctggtgatggagctcatggatgccaacctgtgccaggtgatccagatggagctggaccatgagaggatgagctacctgctgtaccagatgctgtgtggcatcaagcacctgcacagtgctggaatcatccacagagacctgaagccaagcaacattgtggtgaagtctgactgtacactgaagatcctggactttggactggccagaacagccggcacatcttttatgatgacaccatacgtggtgacaagatactacagagcccctgaggtgatcctgggcatgggctacaaggagaacgtggacatctggtctgtgggctgcatcatgggagagatggtgagacacaagatcctgtttcctggaagagactacattgaccagtggaacaaggtgattgagcagctgggcaccccttgtcctgagttcatgaagaagctgcagccaactgtgaggaactatgtggagaacagaccaaagtatgctggcctgaccttccccaagctcttccctgacagcctgtttcctgctgattctgagcacaacaagctgaaggccagccaggccagagacctgctgagcaagatgctggtgattgatcctgccaagagaatctctgtggatgatgccctgcagcacccctacatcaatgtgtggtacgacccagctgaggtggaggccccacctccacagatctatgacaagcagctggatgagagagagcacacaattgaagagtggaaggagctgatctacaaagaagtgatgaactctgaggagaagaccaagaatggagtggtgaagggccagccctctccaagcgcccaggtgcagcaggactacaaggatgatgatgacaaa

SEQ ID NO: 6 - DNA, 738 bp, Artificial Sequence
  GFP protein coding sequence after optimization
atgagcaagggagaggaactgttcacaggagtggtgcccatcctggtggagctggatggagatgtgaatggccagaagttttctgtgtctggggaaggagaaggcgatgccacctatggcaagctgacactgaagttcatctgcaccacagggaagctgcctgtgccctggccaacactggtgaccaccttctcctatggagtccagtgcttcagcagatacccagaccacatgaagcagcatgacttcttcaagagtgccatgcctgagggctatgtgcaggagagaaccatcttctataaggatgatggaaactacaagacaagagctgaggtgaagtttgagggagacaccctggtgaacagaattgagctgaagggcattgacttcaaggaggatggcaacatcctgggccacaagatggagtacaattacaacagccacaatgtgtacatcatggctgataagccaaagaatggaatcaaggtgaacttcaagattagacacaacatcaaagacggatctgtgcagctggctgaccattaccagcagaacacacccattggagatggcccagtgctgctgcccgacaaccactacctgagcacacagtctgccctgagtaaggaccctaatgagaagagggaccacatgattctgctggagtttgtgacagctgctggcatcacccatggcatggatgagctgtacaaggactacaaagatgatgatgacaag

(19) United States
(12) Patent Application Publication
(10) Pub. No.: US 2021/0027858 A1
(43) Pub. Date: Jan. 28, 2021

(54) CODON OPTIMIZATION METHOD BASED ON IMMUNE ALGORITHM

(71) Applicant: Nanjingjinsirui Science & Technology Biology Corp., Nanjing (CN)

(72) Inventors: Long Fan, Nanjing (CN); Yan Sun, Nanjing (CN); Dongming Wu, Nanjing (CN); Lihua Zhang, Nanjing (CN); Zhenyu Liu, Nanjing (CN)

(73) Assignee: Nanjingjinsirui Science & Technology Biology Corp., Nanjing (CN)

(21) Appl. No.: 16/633,910

(22) PCT Filed: Jul. 25, 2018
(86) PCT No.: PCT/CN2018/097040

§ 371 Date: Jan. 24, 2020

(30) Foreign Application Priority Data
Jul. 25, 2017 (CN) 201710611752.5

(51) Int. Cl. G16B 30/00; G16B 20/00

(52) U.S. Cl. CPC G16B 30/00; G16B 20/00

(57) ABSTRACT

A codon optimization method based on an immune algorithm is characterized in that an immune algorithm and a genetic algorithm are successively used to respectively perform local multi-objective optimization and global multi-objective optimization on a protein coding sequence, and then an exhaustive method is used to perform fine adjustment and optimization on the sequence, so as to search the optimal expression sequence to the greatest extent. The present invention not only retains the characteristic of random global parallel search of the genetic algorithm, but also avoids premature convergence to a comparatively great extent to ensure rapid convergence to the global optimal solution. The present invention is the first to combine the advantages of the immune algorithm and the genetic algorithm in accuracy and efficiency to carry out codon optimization through a step-by-step process (local optimization, global optimization, and fine adjustment and optimization respectively in sequence), and proves the high efficiency of the algorithm in codon optimization through example tests.

### TECHNICAL FIELD

The present invention relates to a protein engineering technology, and in particular to a codon optimization method in protein engineering, and specifically to a codon optimization method based on an immune algorithm.

### BACKGROUND

Codon degeneracy refers to the phenomenon that an amino acid can be encoded by multiple different codons during protein translation. The different codons encoding the same amino acid are called synonymous codons. A protein consisting of 200 amino acids in length can generally be encoded by more than 10²⁰ different DNA sequences. In different species, the occurrence frequency of the synonymous codons is different, and such a phenomenon is called codon preference. Codon optimization is mainly based on factors such as the codon preference of a host expression system. On the premise of not changing the amino acid sequence of a protein, a computer algorithm is used to screen out the DNA sequence that can express the protein most efficiently in the host expression system from a large number of DNA coding sequences.

At present, the main factors that are often considered as affecting protein expression in the process of codon optimization include the codon preference of a host cell (commonly used characterization parameters thereof include a codon adaptation index [CAI], a duplex codon preference of a host cell [Codon Context], CBI [Codon Bias Index], ENC [Effective Number of Codon], FOP [Frequency of Optimal Codons], CPP [Codon Preference Parameter], and tAI [tRNA adaptation index]), the number of Hidden Stop Codons, a GC content, a rare codon content, the number of mRNA inhibitory regulatory motif, a mRNA secondary structure (mainly including a hairpin structure and minimum free energy), scoring of key codons and mathematical models in machine learning, a microRNA binding site, a G4 content, and a codon preference of a protein secondary structure (Joshua B. Plotkin & Grzegorz Kudla, Nature Reviews Genetics, 2011). The software and algorithms currently available for codon optimization include DNAWorks, Jcat, Synthetic gene designer, GeneDesign 2.0, OPTIMIZER, Eugene, mRNA Optimizer, COOL, D-Tailor, UpGene, GASCO, Codon Harmonization, QPSO, GeMS and ATGME (Evelina Angov, Biotechnology Journal, 2011; Nathan Gould et al., Frontiers in Bioengineering and Biotechnology, 2014).

Compared with a heuristic algorithm (such as a particle swarm and genetic algorithm) that has been used in codon optimization algorithms, an immune algorithm has its unique advantages. The immune algorithm is an improved genetic algorithm based on a biological immune mechanism. It enables the objective function of an actual problem to be solved to correspond to an antigen and enables the solution of the problem to correspond to an antibody. According to the principle of biological immunity, it can be seen that a biological immune system automatically generates corresponding antibodies through cell division and differentiation to resist antigens that invade living organisms. Such a process is referred to as immune response. In the process of immune response, some antibodies are preserved as memory cells, and when antigens of the same type invade again, the memory cells are activated and produce a large number of antibodies rapidly, which makes the re-response faster and stronger than the initial response, which reflects the memory function of the immune system. After binding with antigens, the antibodies destroy the antigens through a series of reactions. At the same time, different antibodies also promote and inhibit each other to maintain the diversity of antibodies and an immune balance. Such a balance is achieved according to a concentration mechanism, that is, the higher the concentration of antibodies, the more inhibited the antibodies are; and the lower the concentration, the more promoted the antibodies are, reflecting the self-regulation function of the immune system.

### SUMMARY

An objective of the present invention is to solve the problems of long cycle and poor expression accuracy of existing codon optimization methods, and to invent a codon optimization method based on an immune algorithm, which can effectively complete large-scale search of a codon optimization space within a limited time, i.e., screening out the DNA sequence that has the most effective expression from a protein coding sequence set.

The technical solution of the present invention is as follows.

A codon optimization method based on an immune algorithm includes that an immune algorithm and a genetic algorithm are successively used to respectively perform local multi-objective optimization and global multi-objective optimization on a protein coding sequence, and then an exhaustive method is used to perform fine adjustment and optimization on the sequence, so as to search the optimal expression sequence to the greatest extent.

In particular, the method of the present invention includes the following three steps: a first step of local optimization, that is, cleaving the protein sequence into non-overlapping sequence fragments A₁, A₂ . . . Aₙ, and then using the immune algorithm to complete the codon optimization for each sequence fragment, so as to generate an approximately optimal DNA sequence set B₁, B₂ . . . Bₙ; a second step of global optimization, that is, initializing the DNA coding sequence of the full length of the protein based on B₁, B₂ . . . Bₙ utilizing the genetic algorithm, and screening out the optimal DNA sequence C₁ of the protein sequence; and a third step of fine adjustment and optimization, which comprises performing exhaustive optimization on the 5′ terminal of the DNA sequence corresponding to the N-terminal region of the encoded protein to generate a DNA sequence C₂, and eliminating an expression inhibitory motif, to finally generate the optimal expression sequence D.

The protein refers to a compound consisting of more than 20 amino acids; the protein includes a secretory protein, a membrane protein, a cytoplasmic protein, a nuclear protein, etc. in terms of locating; includes an antibody protein, a regulatory protein, a structural protein, etc. in terms of functions; includes a homologous expression protein and a heterologous expression protein in terms of sources; includes a natural protein and an artificially-modified protein, a complete protein/antibody, a truncated partial protein/antibody, and a fusion protein formed from 2 or more proteins and from a protein and a peptide chain in terms of sequences. The antibody defined in the present invention includes, but is not limited to, an intact antibody, and Fab, ScFV, SdAb, a Chimeric antibody, a bispecific antibody, a Fc fusion protein, and the like.

The immune genetic algorithm adopts a multi-objective optimization method to perform local optimization on the protein fragments, the population initialization is based on a duplex codon table of a sequence encoding a highly-expressed protein, and each gene is directly encoded by synonymous codons; and in the optimization process, antibody diversity is ensured and the phenomenon of population degeneration is prevented by calculating antibody information entropy, antibody population similarity, antibody concentration and polymerization fitness of the immune genetic algorithm and updating memory cells, so as to increase the global search capability of the algorithm.

The genetic algorithm adopts the multi-objective optimization method to perform global optimization on the full sequence of the protein, an initialized population is randomly generated based on optimized fragments subjected to local optimization, and each gene is directly encoded by an optimized sequence set of each protein fragment.

The fine adjustment and optimization uses the exhaustive method to calculate and sort the minimum free energy MFE, Codon Context and CAI at the 5′ terminal of the DNA sequence, and selects the optimum coding sequence for the N-terminal of the protein sequence according to the sorting result.

The codon optimization method is at least applicable to the following host expression systems: 1) a mammalian expression system; 2) an insect expression system; 3) a yeast expression system; 4) a Escherichia coli expression system; 5) a Bacillus subtilis expression system; 6) a plant expression system, and 7) a cell-free expression system.

The codon optimization method is at least applicable to the following expression vectors: a transient expression vector and a stable expression vector, a viral expression vector and a non-viral expression vector, induced and non-induced expression vectors.

The beneficial effects of the present invention are as follows.

The immune algorithm is an algorithm improved from the genetic algorithm. In view of the advantage of the immune algorithm in preventing premature local convergence in optimization, the present invention is the first to introduce the immune algorithm to carry out codon optimization for local optimization, and carries out global optimization through the subsequent genetic algorithm and finally carries out fine adjustment and optimization, and thus develops a brand-new three-step hybrid optimization algorithm which combines the advantages of different algorithms; and the high efficiency of the algorithm in codon optimization is further proved by the following Examples.

Compared with the genetic algorithm, the immune algorithm of the present invention has the following characteristics: firstly, the immune algorithm has an immune memory function which can accelerate the search speed and improve the overall search capability of the genetic algorithm; secondly, it has the function of maintaining the diversity of antibodies, which can be utilized to improve the local searching ability of the genetic algorithm; and finally, it has a self-regulating function, which can be used to improve the global search ability of the genetic algorithm and avoid falling into a local solution. Therefore, the immune genetic algorithm not only retains the characteristic of random global parallel search of the genetic algorithm, but also avoids premature convergence to a comparatively great extent to ensure rapid convergence to the global optimal solution. The present invention is the first to combine the advantages of the immune algorithm and the genetic algorithm in accuracy and efficiency to carry out codon optimization through a step-by-step process (local optimization, global optimization, and fine adjustment and optimization respectively in sequence), and proves the high efficiency of the algorithm in codon optimization through example tests.

The present invention has the advantages of high speed and high efficiency.

### BRIEF DESCRIPTION OF DRAWINGS

FIG. 1 is a schematic flow chart of an optimization algorithm of the present invention.

FIG. 2 is a schematic flow chart of an immune algorithm of the present invention (i.e., a local optimization flow).

FIG. 3 shows a flow of a genetic algorithm of the present invention (i.e., a global optimization flow).

FIG. 4 shows a flow of optimizing the 5′ terminal of the DNA sequence of the present invention.

FIG. 5 is a schematic diagram of the gene sequence design of a test protein of the present invention.

FIG. 6 is a pTT expression vector map of the present invention.

FIG. 7 is a schematic diagram of Western Blotting results of the present invention.

### DETAILED DESCRIPTION

The following further describes the present invention with reference to the accompanying drawings and specific examples.

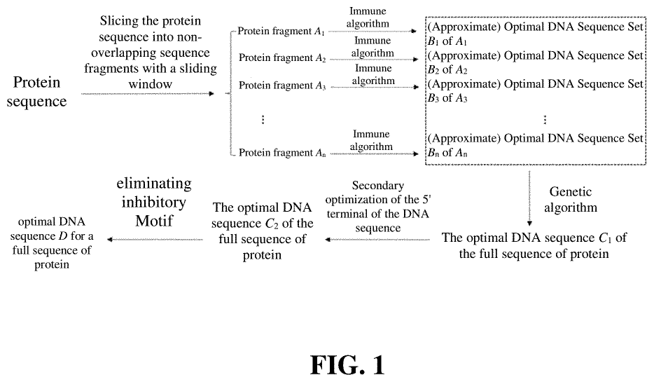

It is as shown in FIGS. 1-7.

A codon optimization method based on an immune algorithm includes that an immune algorithm and a genetic algorithm are successively used to respectively perform local multi-objective optimization and global multi-objective optimization on protein coding sequences (SEQ ID NO. 3 and SEQ ID NO. 4), and then an exhaustive method is used to perform fine adjustment and optimization on the sequence, so as to search the optimal expression sequences (SEQ ID NO. 5 and SEQ ID NO. 6) to the greatest extent, as shown in FIG. 1, wherein:

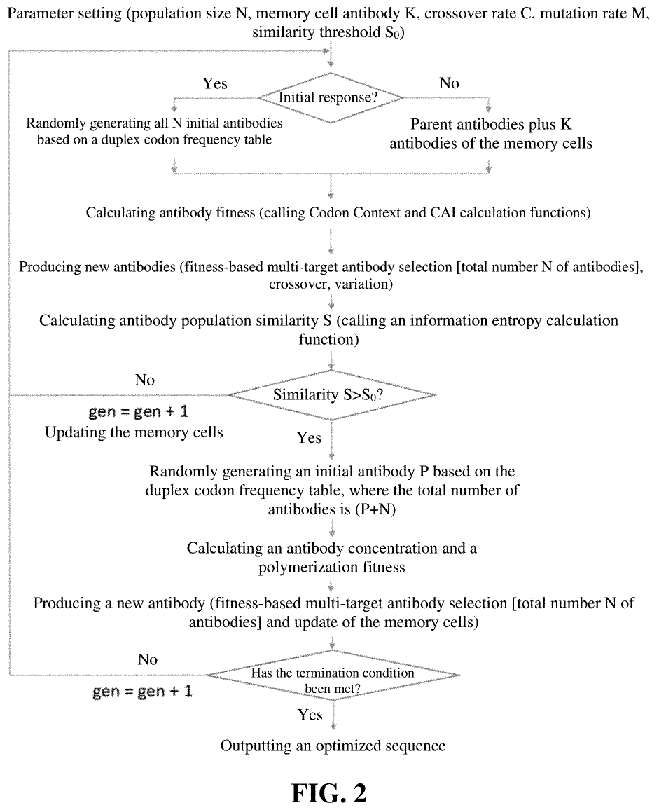

I. Immune Algorithm (i.e., Local Optimization, see FIG. 2 for the Flow).

The number of optimization variables L in this step is 2, that is, two features, Codon Context and CAI, are optimized for each fragment (see below for detailed description), which belongs to multi-objective optimization. Assuming that an immune system consists of N antibodies (i.e., the population size is N), each antibody gene has a length of M (equivalent to that the number of amino acids in the protein sequence is M), and each gene is directly encoded with synonymous codons.

(1) According to a basic data set of different host expression systems (i.e., coding sequences of highly-expressed proteins), a codon frequency table and a duplex codon frequency table are calculated for generating sequences and calculating the codon context and CAI.

(2) In the initial response, an initial antibody is generated according to the duplex codon frequency. Taking the protein sequence a₁a₂ . . . aₘ as an example, it is assumed that the synonymous codons for a₁ are c₁₁ and c₁₂, and the synonymous codons for a₂ are c₂₁, c₂₂ and c₂₃. The codons for the first amino acid a₁ are selected according to the frequencies of c₁₁ and c₁₂ in the codon frequency table. The duplex codons corresponding to the duplex amino acid a₁a₂ are c₁₁c₂₁, c₁₁c₂₂, c₁₁c₂₃, c₁₂c₂₁, c₁₂c₂₂, and c₁₂c₂₃, where there are two sets of duplex synonymous codons, including [c₁₁c₂₁, c₁₁c₂₂, c₁₁c₂₃] and [c₁₂c₂₁, c₁₂c₂₂, c₁₂c₂₃]. Assuming that the codon selected for a₁ is C₁₁, then the codon for the amino acid a₂ is selected from one of c₂₁, c₂₂ and c₂₃ according to the frequencies of c₁₁c₂₁, c₁₁c₂₂ and c₁₁c₂₃. If the codon selected for a₁ is C₁₂, then the codon for the amino acid a₂ is selected from one of c₂₁, c₂₂ and c₂₃ according to the frequencies of c₁₂c₂₁, c₁₂c₂₂ and c₁₂c₂₃. In brief, the selection of codons for other amino acids is related to the selection of codons for its previous amino acid and is determined by the frequency of their duplex synonymous codons, except that the codon for the first amino acid is directly selected according to the codon frequency table.

(3) In a non-initial response, the population consists of parent individuals and K antibodies stored in a memory cell. The antibodies of the memory cell record the K optimum antibodies that have appeared in the optimization history, where the antibodies with low fitness are gradually replaced by individuals with higher fitness in the optimization process.

(4) The fitness F (including F_{[codon Context]} and F_{[CAI]}) of an antibody is calculated, N progeny individuals are selected according to multi-objective optimization, and crossover and variation operations are completed for the new population. The variation here is random mutation of codon.

(5) Calculating of antibody population similarity S

The present invention uses Shannon's average information entropy H(N) to measure the population similarity S.

First, Pᵢⱼ is the probability that a synonymous codon i appears on an amino acid j, namely:

$${P}_{ij} = \frac{{N}_{ij}}{N} ,$$

where Nᵢⱼ is the total number of synonymous codon i that appears at the j-th amino acid position of all individuals in the population. Then Hj(N) is the information entropy of the j-th gene (i.e. the j-th amino acid of the protein sequence), and is defined as:

$${H}_{j} ( N ) = - {\sum }_{i = 1}^{N} {P}_{ij} {log}_{2} {P}_{ij}$$

The average information entropy of the whole population is:

$$H ( N ) = - \frac{1}{M} {\sum }_{j = 1}^{M} {H}_{j} ( N )$$

The population similarity S is defined as:

$$S = \frac{1}{1 + H ( N )}$$

(6) With the progress of optimization, the similarity of antibodies in the population is continuously improved. In order to avoid the homogeneity of antibodies and improve the diversity of antibodies, and thus improve the global search ability and prevent premature convergence, when the population similarity S is greater than the threshold S₀, the metabolic function of the immune system cells is simulated to generate P new antibodies, and the generation process is the same as the above (2), so that the total number of antibodies reaches P+N. If the population similarity S is less than the threshold S₀, then the population continues to directly enter the next generation of evolution and the memory cells are updated.

(7) When S>S₀, the antibody concentration and polymerization fitness are calculated for the antibody population P+N. The antibody concentration refers to the percentage of antibodies similar to each antibody in the population, i.e.,

$${C}_{i} = \frac{{A}_{i}}{N - 1}$$

where Ai refers to the number of antibodies whose similarity to the antibody i is greater than a similarity constant λ. λ refers to the number of identical codons among M codons when two individuals are compared.

Polymerization fitness F′ is a value obtained after the antibody fitness F is corrected according to the antibody concentration, namely:

$${F}_{i}^{′} = α \frac{{F}_{i}}{{\sum }_{i}^{N} {F}_{i}} + ( 1 - α ) \frac{{A}_{i}}{{\sum }_{i}^{N} {A}_{i}} ( 0 < α < 1 )$$

According to the polymerization fitness, a progeny population is selected, and memory cells are updated, and a next round of optimization is carried out. Since we consider the two sequence features, codon context and CAI, at the same time, F′_{[codon context]} is calculated based on F_{[codon context]}, and F′_{[CAI]} is calculated based on F′_{[CAI]}. If a termination algebra is reached, the evolution is stopped, and an optimized sequence set of a single protein fragment is output.

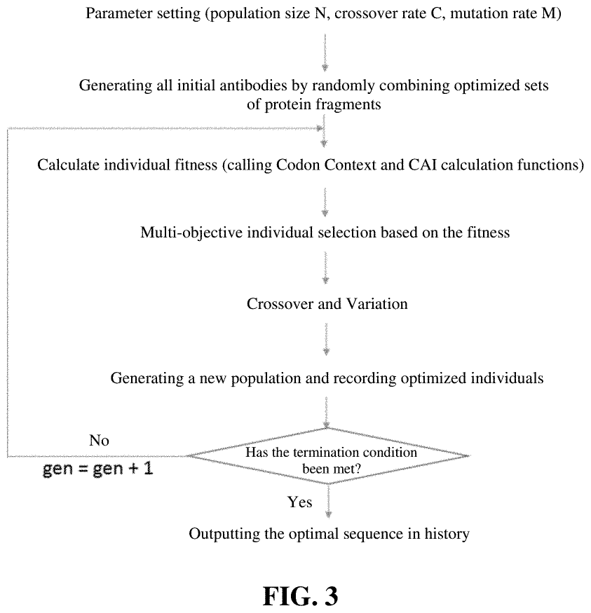

II. Genetic Algorithm (i.e., Global Optimization, see FIG. 3 for the Flow).

Based on the optimized sequence set of all protein fragments generated by the optimization through the immune algorithm, an initialized population N is randomly generated. According to the flow of the genetic algorithm, fitness calculation, selection of a progeny population, crossover, variation and memory update are completed. If a termination algebra is reached, the evolution is stopped, and the optimal DNA coding sequence for the full sequence of the protein is output. The whole flow belongs to the multi-objective optimization. In the optimization process, we directly use the optimized sequence set of each protein fragment to encode each gene.

III. Fine Adjustment and Optimization.

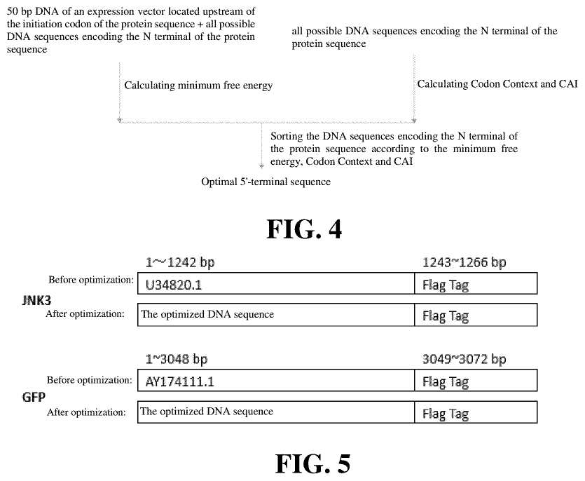

The fine adjustment and optimization consists of two steps: first, optimizing the 5′ terminal of the DNA, and then eliminating the expression inhibitory motif. The optimization process of the 5′ terminal of DNA is as shown in FIG. 4. The exhaustive method is used to list all possible DNA coding sequences of the N-terminal amino acid sequence (8-15 amino acids) of the protein, and to calculate their codon context and CAI. Then 50 bp (with a default value of 50 bp, and a selectable length range of 0-50 bp) of a vector sequence located upstream of an initiation codon of the protein sequence is connected to the DNA coding sequence sequentially, and the minimum free energy (MFE) of the connected sequence is calculated by software mfold. According to the minimum free energy (the greater the value, the better), the codon context (the greater the value, the better) and CAI (the greater the value, the better), the coding sequences of signal peptides are sorted to select the best 5′-terminal sequence.

IV. Details of the Above Flow

(1) Generation of Basic Data Set and Duplex Codon Table

The basic data set refers to highly-expressed proteins in different host expression systems and their corresponding DNA coding sequences. The duplex codon table refers to the relative fitness of all duplex codons in the basic data set (see below for the calculation method).

(2) calculation flow of codon context and CAI

a) codon relative fitness wᵢⱼ:

$${w}_{ij} = \frac{{x}_{ij}}{{x}_{imax}}$$

where xᵢⱼ represents the number of the j-th synonymous codon of the i-th type of amino acid appeared in the basic data set, and xᵢₘₐₓ represents the number of synonymous codons with the highest use frequency for the i-th type of amino acid appeared in the basic data set.

b) Codon Adaptation Index (CAI) of a target sequence:

$$CAI = {( {\prod }_{k = 1}^{L} {w}_{k} )}^{\frac{1}{L}}$$

where L refers to the number of amino acids of the target sequence (i.e., the protein sequence or fragment), wₖ is the codon relative fitness of the basic data set corresponding to the codon used by each amino acid codon. CAI has a value between 0 and 1. In the optimization process, we try our best to increase the CAI value of the encoding DNA.

c) Relative fitness pₖ of duplex codon:

$${p}_{k} = \frac{{α}_{CC}^{k}}{{α}_{A A}^{j ( k )}} , ∀ k ⊆ \{ 1 , 2 , … , 3721 \}$$

where there are 3,721 kinds of duplex codons (^{61×61=3721}, without considering termination codons), a_{CC}^{k} represents the number of the k-th type of duplex codons appeared in the protein sequence basic data set or the target sequence (i.e., the protein sequence or a fragment thereof), and a_{AA}^{J(k)} represents the number of duplex amino acids corresponding to the duplex codons as appeared.

d) Codon Context (CC) of the target sequence:

$$C C = 1 - \frac{{\sum }_{k = 1}^{3721} \vert {p}_{0}^{k} - {p}_{1}^{k} \vert}{3721}$$

where P₀^{k} represents the relative fitness of the k-th type of duplex codons of the target sequence, and P₁^{k} represents the relative fitness of the k-th type of duplex codons of the basic data set. CC has a value between 0 and 1. In the optimization process, we try our best to increase the CC value of the encoding DNA.

(3) NSGA2 and SPEA2 algorithms (NSGA2 is used by default) can be used for selection of progeny population in the multi-objective optimization process of the immune algorithm and the genetic algorithm, and two-point crossover is used for crossover.

The following further illustrates the advantages of the present invention by an example.

The host expression system used in the test is a CHO cell line, and two proteins are optimized and sequenced in total (see Table 1 for relevant information). The JNK3 protein sequence is as shown in SEQ ID NO. 1, and the GFP protein sequence is as shown in SEQ ID NO. 2; the coding sequences of the JNK3 protein and the GFP protein before optimization are as shown in SEQ ID NO. 3 and SEQ ID NO. 4 respectively, and the coding sequences of the JNK3 protein and the GFP protein after optimization are as shown in SEQ ID NO. 5 and SEQ ID NO. 6 respectively.

TABLE 1
Information of Optimized Test Protein Sequences

| | GenBank accession | | |
|---|---|---|---|
| Protein | number (Wild Type) | tag | Position of Tag |
| JNK3 | U34820.1 | Flag tag | C terminal |
| GFP | AY174111.1 | Flag tag | C terminal |

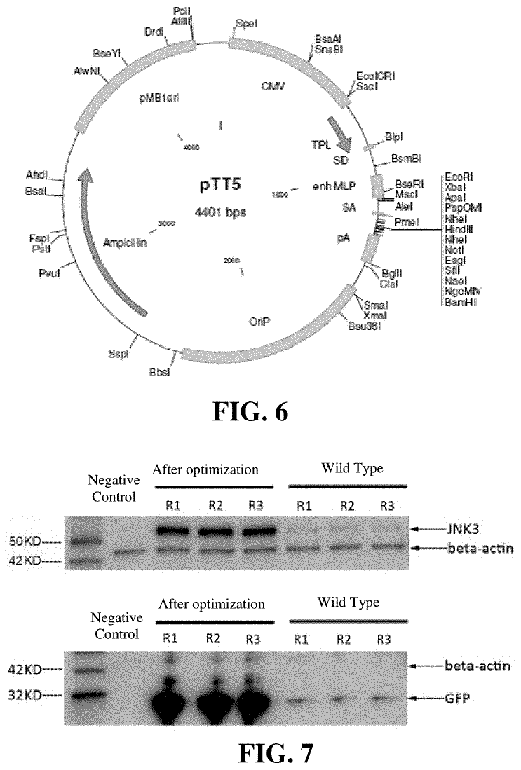

As shown in FIG. 5, the gene fragment encoding the test protein is synthesized and cloned into a pTT5 expression vector (purchased from NRC, and the plasmid map is as shown in FIG. 6) via EcoR I and Hind III cleavage sites respectively.

Transient Expression Steps of CHO 3E7 Cells:

1. CHO 3E7 suspension cells in the logarithmic growth phase are diluted to 5×10⁵ cells/mL with a fresh FreeStyle CHO medium, and 30 mL of a cell suspension is inoculated in each 125 mL triangular flask.

2. The cells are subjected to suspension culture under conditions of 37° C. and 5% CO₂.

3. When the cell density reaches 1-1.2×10⁶ cells/mL, plasmid vectors carrying cloned target genes are transfected into CHO 3E7 cells respectively according to the dosage of 1 ug/ml by a PEI transfection reagent.

4. After 48 hours of transfection, the culture medium is centrifuged at 1500 rotations/min to harvest cells. The samples can be stored in a refrigerator at −80° C.

Western Blot Experiment Steps:

Using an anti-Flag tag antibody, the expression quantity of the target protein in a cell lysate was detected by Western Blotting. A beta-actin protein is used as internal reference. The expression experiment of each plasmid is replicated for three times. The results of Western Blotting are shown in FIG. 7.

The detailed steps are as follows.

1. CHO cells are lysed using a cell lysis buffer, and the protein concentration is determined.

2. The protein solution is added with a 5× SDS-PAGE protein loading buffer, and heated in a boiling water bath for 10 minutes.

3. The protein sample is added into sample loading wells of an SDS-PAGE gel with a micropipette, and each well is loaded with 20 ul of the sample.

4. A constant voltage electrophoresis at 140 V is used for 60 minutes, and the electrophoresis is stopped when bromophenol blue reaches near the bottom of the gel.

5. The membrane transfer voltage is 100 V, and the membrane transfer time at low temperature is 60 minutes.

6. After the membrane transfer is completed, the protein membrane is placed in a washing liquid prepared in advance, and rinsed for 1-2 minutes to remove the membrane transfer liquid on the membrane.

7. It is blocked by slowly shaking on a shaker at room temperature for 45 minutes.

8. It is added with a diluted primary antibody and incubated at room temperature for one hour with slow shaking.

9. It is added with the washing liquid, and slowly shaken for washing on the shaker for 5 minutes for 3 times in total.

10. It is added with a diluted secondary antibody and incubated at room temperature for one hour with slow shaking.

11. It is added with the washing liquid, and slowly shaken for washing on the shaker for 5 minutes for 3 times in total.

12. Chemiluminescence detection.

13. The Western Blotting result picture is quantitatively analyzed with software Image J.

TABLE 2
Relative Expression Quantity of Protein Before and After
Optimization (As Detected by Western Blotting)

| | GFP (relative | JNK3 (relative expression |
|---|---|---|
| | expression quantity ± | quantity ± standard |
| | standard deviation) | deviation) |
| After Optimization | 22.06 ± 1.78 | 8.01 ± 0.21 |
| Wild Type | 1.19 ± 0.16 | 1.09 ± 0.10 |
| Ratio | 18.37 ± 2.90 | 7.42 ± 0.58 |

*Relative expression quantity: a protein expression quantity divided by the minimum value of expression quantity in three replicated experiments of wild-type sequences

As can be seen from Table 2, the expression quantities of JNK3 and GFP proteins after being subjected to the three-step hybrid codon optimization of this patent are respectively increased by 7.42±0.58 times and 18.37±2.90 times compared with that of the wild-type sequence, which fully proves the high efficiency of the new algorithm. In the actual production of a company, we also compare and test the optimization effects of this algorithm and other algorithms on multiple proteins, which also proves that this algorithm is more stable and efficient.

The parts not involved in the present invention are all the same as those in the prior art or can be realized by using the prior art.

1. A codon optimization method based on an immune algorithm, wherein an immune algorithm and a genetic algorithm are successively used to respectively perform local multi-objective optimization and global multi-objective optimization on a protein coding sequence, and then an exhaustive method is used to perform fine adjustment and optimization on the sequence, so as to search the optimal expression sequence to the greatest extent.

2. The optimization method according to claim 1, comprising the following three steps: a first step of local optimization, that is, cleaving the protein sequence into non-overlapping sequence fragments A₁, A₂ . . . Aₙ, and then using the immune algorithm to complete the codon optimization for each sequence fragment, so as to generate an approximately optimal DNA sequence set B₁, B₂ . . . Bₙ; a second step of global optimization, that is, initializing the DNA coding sequence of the full length of the protein based on B₁, B₂ . . . Bₙ utilizing the genetic algorithm, and screening out the optimal DNA sequence C₁ of the protein sequence; and a third step of fine adjustment and optimization, which comprises performing exhaustive optimization on the 5′ terminal of the DNA sequence corresponding to the N-terminal region of the encoded protein to generate a DNA sequence C₂, and eliminating an expression inhibitory motif, to finally generate the optimal expression sequence D.

3. The optimization method according to claim 1, wherein the protein refers to a compound consisting of more than 20 amino acids; the protein comprises a secretory protein, a membrane protein, a cytoplasmic protein, a nuclear protein, etc. in terms of locating; comprises an antibody protein, a regulatory protein, a structural protein, etc. in terms of functions; comprises a homologous expression protein and a heterologous expression protein in terms of sources; comprises a natural protein and an artificially-modified protein, a complete protein/antibody, a truncated partial protein/antibody, and a fusion protein formed from 2 or more proteins and from a protein and a peptide chain in terms of sequences; the antibody defined in the present invention comprises, but is not limited to, an intact antibody, and Fab, ScFV, SdAb, a chimeric antibody, a bispecific antibody, a Fc fusion protein, and the like.

4. The optimization method according to claim 1, wherein the immune genetic algorithm adopts a multi-objective optimization method to perform local optimization on the protein fragments, the population initialization is based on a duplex codon table of a sequence encoding a highly-expressed protein, and each gene is directly encoded by synonymous codons; and in the optimization process, antibody diversity is ensured and the phenomenon of population degeneration is prevented by calculating antibody information entropy, antibody population similarity, antibody concentration and polymerization fitness of the immune genetic algorithm and updating memory cells, so as to increase the global search capability of the algorithm.

5. The optimization method according to claim 1, wherein the genetic algorithm adopts the multi-objective optimization method to perform global optimization on the full sequence of the protein, an initialized population is randomly generated based on optimized fragments subjected to local optimization, and each gene is directly encoded by an optimized sequence set of each protein fragment.

6. The optimization method according to claim 1, wherein the fine adjustment and optimization uses the exhaustive method to calculate and sort the minimum free energy MFE, Codon Context and CAI at the 5′ terminal of the DNA sequence, and selects the optimum coding sequence for the N-terminal of the protein sequence according to the sorting result.

7. The optimization method according to claim 1, wherein the codon optimization method is at least applicable to the following host expression systems: 1) a mammalian expression system; 2) an insect expression system; 3) a yeast expression system; 4) a Escherichia coli expression system; 5) a Bacillus subtilis expression system; 6) a plant expression system, and 7) a cell-free expression system.

8. The optimization method according to claim 1, wherein the codon optimization method is at least applicable to the following expression vectors: a transient expression vector and a stable expression vector, a viral expression vector and a non-viral expression vector, induced and non-induced expression vectors.

9. The optimization method according to claim 2, wherein the protein 5 refers to a compound consisting of more than 20 amino acids; the protein comprises a secretory protein, a membrane protein, a cytoplasmic protein, a nuclear protein, etc. in terms of locating; comprises an antibody protein, a regulatory protein, a structural protein, etc. in terms of functions; comprises a homologous expression protein and a heterologous expression protein in terms of sources; comprises a natural protein and an artificially-modified protein, a complete protein/antibody, a truncated partial protein/antibody, and a fusion protein formed from 2 or more proteins and from a protein and a peptide chain in terms of sequences; the antibody defined in the present invention comprises, but is not limited to, an intact antibody, and Fab, ScFv, SdAb, a chimeric antibody, a bispecific antibody, a Fc fusion protein, and the like.

10. The optimization method according to claim 2, wherein the immune genetic algorithm adopts a multi-objective optimization method to perform local optimization on the protein fragments, the population initialization is based on a duplex codon table of a sequence encoding a highly-expressed protein, and each gene is directly encoded by synonymous codons; and in the optimization process, antibody diversity is ensured and the phenomenon of population degeneration is prevented by calculating antibody information entropy, antibody population similarity, antibody concentration and polymerization fitness of the immune genetic algorithm and updating memory cells, so as to increase the global search capability of the algorithm.

11. The optimization method according to claim 2, wherein the genetic algorithm adopts the multi-objective optimization method to perform global optimization on the full sequence of the protein, an initialized population is randomly generated based on optimized fragments subjected to local optimization, and each gene is directly encoded by an optimized sequence set of each protein fragment.

12. The optimization method according to claim 2, wherein the fine adjustment and optimization uses the exhaustive method to calculate and sort the minimum free energy MFE, Codon Context and CAI at the 5′ terminal of the DNA sequence, and selects 5 the optimum coding sequence for the N-terminal of the protein sequence according to the sorting result.

13. The optimization method according to claim 2, wherein the codon optimization method is at least applicable to the following host expression systems: 1) a mammalian expression system; 2) an insect expression system; 3) a yeast expression system; 4) a Escherichia coli expression system; 5) a Bacillus subtilis expression system; 6) a plant expression system, and 7) a cell-free expression system.

14. The optimization method according to claim 2, wherein the codon optimization method is at least applicable to the following expression vectors: a transient expression vector and a stable expression vector, a viral expression vector and a non-viral expression vector, induced and non-induced expression vectors.
